(19) United States
(12) Patent Application Publication
(10) Pub. No.: US 2020/0016435 A1
(43) Pub. Date: Jan. 16, 2020

(54) NERVE MODULATION DEVICES AND RELATED METHODS

(71) Applicant: ELWHA LLC, Bellevue, WA (US)

(72) Inventors: Jeffrey A. Bowers, Bellevue, WA (US); Paul Duesterhoft, Grapevine, TX (US); Daniel Hawkins, Pleasanton, CA (US); Roderick A. Hyde, Redmond, WA (US); Edward K.Y. Jung, Las Vegas, NV (US); Jordin T. Kare, San Jose, CA (US); Eric C. Leuthardt, St. Louis, MO (US); Nathan P. Myhrvold, Bellevue, WA (US); Jay Pierce, Cutler Bay, FL (US); Katherine E. Sharadin, Redmond, WA (US); Michael A. Smith, Phoenix, AZ (US); Marc Stein, Phoenix, AZ (US); Elizabeth A. Sweeney, Seattle, WA (US); Clarence T. Tegreene, Mercer Island, WA (US); Lowell L. Wood, JR., Bellevue, WA (US)

(21) Appl. No.: 16/032,623

(22) Filed: Jul. 11, 2018

(51) Int. Cl. A61N 7/00; A61B 5/00

(52) U.S. Cl. CPC A61N 7/00; A61N 1/36528; A61B 5/6822

(57) ABSTRACT

A nerve modulation device includes a first ultrasound transducer and a second ultrasound transducer. The first and second ultrasound transducers are configured to emit a first and second ultrasound waves, respectively, that exhibit different frequencies. The first and second ultrasound transducers can emit the first and second ultrasound waves in directions that are selected to cause the first and second ultrasound waves to intersect with each other at an intersection site that is at or near a selected nerve. At the intersection site, the first and second ultrasound waves can non-linearly interact to form an acoustic wave exhibiting a frequency that is less than the frequencies of the first and second ultrasound waves. The acoustic wave can modulate a selected nerve.

If an Application Data Sheet (ADS) has been filed on the filing date of this application, it is incorporated by reference herein. Any applications claimed on the ADS for priority under 35 U.S.C. §§ 119, 120, 121, or 365(c), and any and all parent, grandparent, great-grandparent, etc. applications of such applications, are also incorporated by reference, including any priority claims made in those applications and any material incorporated by reference, to the extent such subject matter is not inconsistent herewith.

### CROSS-REFERENCE TO RELATED APPLICATIONS

The present application claims the benefit of the earliest available effective filing date(s) from the following listed application(s) (the “Priority Applications”), if any, listed below (e.g., claims earliest available priority dates for other than provisional patent applications or claims benefits under 35 USC § 119(e) for provisional patent applications, for any and all parent, grandparent, great-grandparent, etc. applications of the Priority Application(s)).

### Priority Applications

None

If the listings of applications provided above are inconsistent with the listings provided via an ADS, it is the intent of the Applicant to claim priority to each application that appears in the Domestic Benefit/National Stage Information section of the ADS and to each application that appears in the Priority Applications section of this application.

All subject matter of the Priority Applications and of any and all applications related to the Priority Applications by priority claims (directly or indirectly), including any priority claims made and subject matter incorporated by reference therein as of the filing date of the instant application, is incorporated herein by reference to the extent such subject matter is not inconsistent herewith.

### BACKGROUND

Nerve modulation has been used to treat several disorders. For example, stimulating certain nerves has been found to cause the nerves to detect higher than actual blood pressure which, in turn, causes the body to reduce the blood pressure.

Therefore, users and manufacturers of nerve modulation devices continue to seek new nerve modulation devices and new methods of using nerve modulation devices.

### SUMMARY

In an embodiment, a nerve modulation device is disclosed. The nerve modulation device includes a first ultrasound transducer configured to emit a first ultrasound wave exhibiting a first frequency. The first ultrasound transducer is positionable on or in a subject. The first ultrasound transducer is configured to emit the first ultrasound wave in a first direction when the first ultrasound transducer is positioned on or in the subject. The nerve modulation device also includes a second ultrasound transducer configured to emit a second ultrasound wave exhibiting a second frequency that is different than the first frequency. The second ultrasound transducer is positionable on or in the subject. The second ultrasound transducer is configured to emit the second ultrasound wave in a second direction when the second ultrasound transducer is positioned on or in the subject. The second direction is selected intersect the second ultrasound wave with the first ultrasound wave at an intersection site at or near a selected nerve. Further, the nerve modulation device includes a controller operably coupled to the first ultrasound transducer and the second ultrasound transducer. The controller is configured to direct the first ultrasound transducer and the second ultrasound transducer to selectively and controllably emit the first ultrasound wave and the second ultrasound wave.

In an embodiment, a method to modulate activity of a selected nerve of a subject is disclosed. The method includes emitting a first ultrasound wave in a first direction from a first ultrasound transducer, the first ultrasound wave exhibiting a first frequency. The method also includes emitting a second ultrasound wave in a second direction from a second ultrasound transducer. The second ultrasound wave exhibiting a second frequency that is different than the first frequency. The method further includes intersecting the first ultrasound wave and the second ultrasound wave at an intersection site that is within the subject and at or near a selected nerve. Additionally, the method includes, responsive to intersecting the first ultrasound wave and the second ultrasound wave, non-linearly interacting the first ultrasound wave with the second ultrasound wave to form an acoustic wave having a frequency that is less than the first frequency and the second frequency. Further, the method includes exposing the selected nerve of the subject to the acoustic wave.

In an embodiment, a method to modulate activity of a selected nerve of a subject is disclosed. The method includes positioning a first ultrasound transducer and a second ultrasound transducer against an external surface of the subject. The method also includes detecting one or more characteristics of the subject with at least one sensor. The method further includes, responsive to detecting the one or more characteristics of the subject, transmitting one or more sensing signals from the at least one sensor and receiving the one or more sensing signals at a controller. Additionally, the method includes, responsive to receiving the one or more sensing signals at the controller, under the direction of the controller: emitting a focused first ultrasound wave in a first direction from the first ultrasound transducer, emitting a second focused ultrasound wave in a second direction from the second ultrasound transducer, and intersecting the first focused ultrasound wave and the second focused ultrasound wave at an intersection site that is in the subject. The first focused ultrasound wave exhibits a first frequency and the second focused ultrasound wave exhibits a second frequency that is different than the first frequency. Further, the method includes, responsive to intersecting the first focused ultrasound wave and the second focused ultrasound wave, non-linearly interacting the first focused ultrasound wave with the second focused ultrasound wave to form an acoustic wave having a frequency that is less than the first frequency and the second frequency. The method also includes exposing the selected nerve of the subject to the acoustic wave.

Features from any of the disclosed embodiments can be used in combination with one another, without limitation. In addition, other features and advantages of the present disclosure will become apparent to those of ordinary skill in the art through consideration of the following detailed description and the accompanying drawings.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1 is a schematic view of a nerve modulation device, according to an embodiment.

FIG. 2 is a schematic view of a nerve modulation device that is configured to have an intersection site of a first ultrasound wave and second ultrasound wave that is near a selected nerve, according to an embodiment.

FIG. 3 is a schematic view of a nerve modulation device, according to an embodiment.

FIGS. 4A-4D are schematics of at least a portion of different nerve modulation devices that each include at least one attachment device configured to couple the ultrasound transducers of the different nerve modulation devices to the external surface of the subject, according to different embodiments.

FIG. 5 is a schematic view of a nerve modulation device that includes one or more components implanted in the subject, according to an embodiment.

FIG. 6 is a schematic view of a nerve modulation device that includes three ultrasound transducers, according to an embodiment.

FIGS. 7A-7B are schematic cross-sectional views of an ultrasound transducer that is configured change a direction that the ultrasound transducer emits ultrasound waves, according to an embodiment.

FIG. 8A is a schematic illustration of an ultrasound array that can be used in any of the ultrasound transducers disclosed herein, according to an embodiment.

FIG. 8B is a schematic view of ultrasound transducers, according to an embodiment.

FIG. 8C is a schematic view of a nerve modulation device that is configured to emit at least one unfocused and uniform ultrasound wave, according to an embodiment.

FIG. 9 is a flow diagram of a method of using any of nerve modulation devices disclosed herein, according to an embodiment.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

Embodiments for nerve modulation devices disclosed herein include first and second ultrasound transducers. The first and second ultrasound transducers are configured to emit first and second ultrasound waves, respectively, that exhibit different frequencies. The first and second ultrasound transducers can emit the first and second ultrasound waves in directions that are selected to cause the first and second ultrasound waves to intersect with each other at an intersection site that is at or near a selected nerve. Unlike audible sound waves, the ultrasound waves emitted by the ultrasound transducers can be focused on the intersection site. At the intersection site, the first and second ultrasound waves can non-linearly interact with each other since the first and second ultrasound waves exhibit different frequencies. For example, the ultrasound waves can non-linearly interact with each other to form an acoustic wave exhibiting a frequency that is less than the frequencies of the first and second ultrasound waves. The acoustic wave can modulate the activity of the selected nerve. The acoustic wave can be more effective than the first and second ultrasound waves at modulating the selected nerve due to the lower frequency thereof. Additionally, the reduced ability to focus the acoustic wave relative to the ultrasound waves is substantially negated since the intersection site is at or near the selected nerve. “Modulate” or “modulating” or “modulation” can include altering the activity of the selected nerve by either enhancing, stimulating or down regulating the activity of the nerve (e.g., excitatory or inhibitory effect on the nerve). Modulation of the selected nerve can result in a complete or partial blocking of the nerve signaling activity. In an embodiment, the acoustic wave can be configured to modulate a nerve of the autonomic nervous system which can, for example, cause the acoustic wave to controllably and selectively affect blood pressure, heart rate, etc. of a subject. In an embodiment, the acoustic wave can be configured to modulate a nerve of the somatic nervous system which can, for example, cause the acoustic wave to controllably and selectively affect one or more of tremors, loss of balance, or other ailment of the subject.

In an embodiment, the nerve modulation devices disclosed herein include a controller operably coupled to the first and second ultrasound transducers. The controller can at least partially control the operation of the first and second ultrasound transducers. The nerve modulation devices disclosed herein can also include one or more sensors coupled to the controller. The sensors can detect one or more characteristics, such as one or more characteristics of the subject, the first or second ultrasound source transducer, the first or second ultrasound wave, or the acoustic wave. The nerve modulation device can also include a user interface coupled to the controller that allows an individual (e.g., the subject, a medical practitioner, etc.) to input information or directions into the controller. The controller can at least partially control the operation of the first and second ultrasound transducers responsive to sensor detecting one or more characteristics detected or receiving the information or instructions from the user interface. For example, the controller can change one or more characteristics of the first or second ultrasound waves (e.g., frequency, direction, amplitude, pulse duration, pulse sequence, etc.) when the sensors or information or instructions from the user interface indicate that the nerve modulation device is not correctly simulating the nerves.

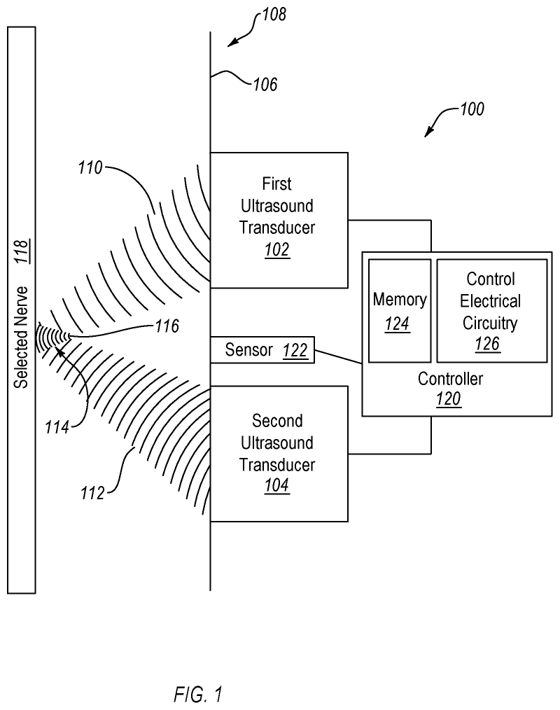

FIG. 1 is a schematic view of a nerve modulation device 100, according to an embodiment. The nerve modulation device 100 includes a first ultrasound transducer 102 and a second ultrasound transducer 104. The first and second ultrasound transducers 102, 104 can be positionable on an external surface 106 (e.g., skin) of a subject 108. The first ultrasound transducer 102 is configured to emit a first ultrasound wave 110 exhibiting a first frequency and the second ultrasound transducer 104 is configured to emit a second ultrasound wave 112 exhibiting a second frequency that is different than the first frequency. The first and second frequencies can be selected to cause the first and second ultrasound waves 110, 112 to non-linearly interact with each other to form an acoustic wave 114 exhibiting a frequency that is less than the first and second frequency. In the illustrated embodiment, the first ultrasound transducer 102 emits the first ultrasound wave 110 in a first direction and the second ultrasound transducer 104 emits the second ultrasound wave 112 in a second direction. The first and second directions can be selected to intersect the first and second ultrasound waves 110, 112 at an intersection site 116 that is at a selected nerve 118. This can cause the selected nerve 118 to be exposed substantially only or preferentially to the acoustic wave 114 (e.g., the selected nerve 118 is only incidentally exposed to the first and second ultrasound waves 110, 112). As such, the acoustic wave 114 can modulate the selected nerve 118 while limiting potential damage of the selected nerve 118 by the first and second ultrasound waves 110, 112. The nerve modulation device 100 can further include a controller 120 that can at least partially controls the first and second ultrasound transducers 102, 104. Additionally, the nerve modulation device 100 can include at least one sensor 122 that is configured to detect one or more characteristics.

The first and second ultrasound transducers 102, 104 can include any suitable ultrasound transducer, such as at least one of a piezoelectric ultrasound transducer or a capacitive ultrasonic transducer. In an embodiment, at least one of the first or second ultrasound transducers 102, 104 can include the piezoelectric ultrasound transducer when the first or second ultrasound transducer 102, 104 has a small size requirement, requires precise control, or lower costs. In an embodiment, at least one of the first or second ultrasound transducers 102, 104 can include the capacitive ultrasonic transducer when greater bandwidth is required.

The first and second ultrasound transducers 102, 104 can be configured to emit ultrasound waves exhibiting a frequency that is greater than about 20 kHz. For example, the first and second ultrasound transducers 102, 104 can be configured to emit ultrasound waves exhibiting a frequency of about 20 kHz to about 50 kHz, about 25 kHz to about 75 kHz, about 50 kHz to about 100 kHz, about 75 kHz to about 150 kHz, about 100 kHz to about 300 kHz, about 250 kHz to about 500 kHz, about 400 kHz to about 800 kHz, about 600 kHz to about 1 MHz, about 750 MHz to about 1.1 MHz, about 1 MHz to about 1.2 MHz, about 1.1 MHz to about 1.4 MHz, about 1.3 MHz to about 1.5 MHz, about 1.4 MHz to about 1.75 MHz, about 1.5 MHz to about 2 MHz, about 1.75 MHz to about 2.5 MHz, about 2 MHz to about 3 MHz, about 2.5 MHz to about 4 MHz, about 3 MHz to about 5 MHz, about 4 MHz to about 7 MHz, about 5 MHz to about 10 MHz, about 7.5 MHz to about 15 MHz, about 10 MHz to about 20 MHz, or greater than about 17 MHz. The frequency that the first and second ultrasound transducers 102, 104 are configured to emit can depend on several different factors. For example, at least one of the first or second ultrasound waves 110, 112 pass through one or more organs that are more susceptible to damage from sound waves as the frequency of the sound waves increase. In such an embodiment, a corresponding one of the first and second ultrasound transducers 102, 104 can be configured to emit relatively low frequency ultrasound waves (e.g., less than about 500 kHz). In an embodiment, at least one of the first or second ultrasound waves 110, 112 pass through one or more organs that are resistant to damage from relatively high frequency ultrasound waves (e.g., greater than about 500 kHz, greater than about 1 MHz). In such an embodiment, a corresponding one of the first and second ultrasound transducers 102, 104 is configured can emit the relatively high frequency ultrasound waves since the relatively high frequency ultrasound waves can be easier to focus that relatively low frequency ultrasound waves.

The first ultrasound wave 110 exhibits a first frequency and the second ultrasound wave 112 exhibits a second frequency that is different than the first frequency. Since the first and second frequencies are different, the first and second ultrasound waves 110, 112 can non-linearly interact with each other to form the acoustic wave 114. For example, the first and second ultrasound waves 110, 112 can produce beats which, in turn, can form the acoustic wave 114. The frequency of the acoustic wave 114 can depend on the difference between the first and second frequencies. For example, the difference between the first and second frequencies can be greater than about 1 Hz, such as about 1 Hz to about 50 Hz, about 25 Hz to about 75 Hz, about 50 Hz to about 100 Hz, about 75 Hz to about 150 Hz, about 100 Hz to about 500 Hz, about 250 Hz to about 750 Hz, about 500 Hz to about 1 kHz, about 750 Hz to about 1.5 kHz, about 1 kHz to about 5 kHz, about 2.5 kHz to about 7.5 kHz, about 5 kHz to about 10 kHz, about 7.5 kHz to about 25 kHz, about 10 kHz to about 50 kHz, about 25 kHz to about 75 kHz, about 50 kHz to about 100 kHz, or greater than about 75 kHz.

In an embodiment, the difference between the first and second frequencies is selected to form an acoustic wave exhibiting a selected frequency. For example, the difference in the frequencies of the first and second ultrasound waves 110, 112 may correspond to the frequency of the acoustic wave 114. That is, a relatively small difference between the first and second frequencies can cause the first and second ultrasound waves 110, 112 to generate an acoustic wave 114 exhibiting a relatively low frequency and a relatively large different between the first and second frequencies can cause the first and second ultrasound waves 110, 112 to generate an acoustic wave 114 exhibiting a relatively large frequency. As such, the difference between the first and second frequencies can be selected based on the desired frequency of the acoustic wave 114.

The frequency of the acoustic wave 114 is less than the first and second frequency frequencies. In an embodiment, the frequency of the acoustic wave 114 can be an audible sound wave exhibiting a frequency of about 20 Hz to about 20 kHz, about 20 Hz to about 50 Hz, about 40 Hz to about 100 Hz, about 75 Hz to about 250 Hz, about 100 Hz to about 500 Hz, about 250 Hz to about 750 Hz, about 500 Hz to about 1 kHz, about 750 Hz to about 2.5 kHz, about 1 kHz to about 5 kHz, about 2.5 kHz to about 7.5 kHz, about 5 kHz to about 10 kHz, or about 7.5 kHz to about 20 kHz. In an embodiment, the frequency of the acoustic waves 114 can be a low ultrasound wave exhibiting a frequency of about 20 kHz to about 1 MHz, about 20 kHz to about 75 kHz, about 50 kHz to about 100 kHz, about 75 kHz to about 250 kHz, about 100 kHz to about 500 kHz, about 250 kHz to about 750 kHz, and about 500 kHz to about 1 MHz. In an embodiment, the frequency of the acoustic wave 114 can be a infrasound wave exhibiting a frequency of about 1 Hz to about 20 Hz, about 2 Hz to about 5 Hz, about 2.5 Hz to about 7.5 Hz, about 5 Hz to about 10 Hz, about 7.5 Hz to about 12.5 Hz, about 10 Hz to about 15 Hz, about 12.5 Hz to about 17.5 Hz, or about 15 Hz to about 20 Hz.

In an embodiment, the acoustic wave 114 can be selected based on a desired interaction with the selected nerve. The desired interaction with the selected nerve 118 can be an excitatory effect (e.g., excitatory synapse) or inhibitory effect (e.g., inhibitory postsynaptic potential). For example, when the modulated nerve is a baroreceptor, the acoustic wave 114 can be selected to have an excitatory effect when the subject 108 has high blood pressure or selected to have an inhibitory effect when the subject 108 has low blood pressure. In an embodiment, the acoustic wave 114 can be selected to have an inhibitory effect when the selected nerve 118 detects or transmits pain signals and the subject 108 is in pain.

In an embodiment, the acoustic wave 114 can be selected based on the selected nerve 118 since the interaction between the acoustic wave 114 and the selected nerve 118 can depend on the particular nerve that is exposed to the acoustic wave 114. For example, the subject 108 can include a first nerve and a second nerve. The first nerve can be a completely different nerve than the second nerve or the first and second nerves can be different portions of the same nerve. In an embodiment, the acoustic wave 114 can have different interactions on the first and second nerve. The different interactions can include at least one of the first nerve exhibit an excitatory effect and the second nerve exhibiting an inhibitory effect, the first nerve exhibiting an excitatory effect and the second nerve exhibiting a greater or lesser excitatory effect, the first nerve exhibiting an inhibitory effect and the second nerve exhibiting a greater or less inhibitory effect, or the first nerve exhibiting an interaction (e.g., an excitatory or inhibitory effect) and the second nerve exhibiting no effect. In an embodiment, changing one or more characteristics (e.g., frequency, amplitude, pulse duration, pulse sequence) of the acoustic wave 114 can have different effects on the first and second nerve.

In an embodiment, the first or second ultrasound transducer 102, 104 can controllably change one or more characteristics of the first or second ultrasound wave 110, 112, respectively. In an embodiment, the first or second ultrasound transducer 102, 104 can controllably change the frequency of the first or second ultrasound wave 110, 112. Changing the frequency of the first or second ultrasound wave 110, 112 can change the frequency of the acoustic wave 114. Changing the frequency of the acoustic wave 114 can change the interaction that the acoustic wave 114 has on the selected nerve 118. In an embodiment, the first or second ultrasound transducer 102, 104 can controllably change the directionality of the first or second ultrasound wave 110, 112. Changing the directionality of the first or second ultrasound wave 110, 112 can cause the acoustic wave to interact with a different portion of the selected nerve 118, cause the acoustic wave to interact with a nerve other than the selected nerve 118, or cause the first and second ultrasound waves 110, 112 to form the acoustic wave 114 when the first and second ultrasound waves 110, 112 to not intersect (e.g., the first or second ultrasound transducers 102, 104 are not correctly positioned on the subject 108 or the first or second ultrasound transducers 102, 104 are inadvertently moved). In an embodiment, the first or second ultrasound transducer 102, 104 can controllably change the amplitude of the first or second ultrasound wave 110, 112. Changing the amplitude of the first or second ultrasound wave 110, 112 can include changing the amplitude of the acoustic wave 114. Changing the amplitude of the acoustic wave 114 can change the interaction between the acoustic wave 114 and the selected nerve 118. It is noted that changing one or more characteristics of the first or second ultrasound wave 110, 112 and, in turn, changing one or more characteristics of the acoustic wave 114 can inhibit nerve accommodation. For example, exposing the selected nerve 118 to the acoustic wave 114 for a prolonged period of time or exposing the selected nerve 118 to the acoustic wave 114 over a number of treatments can cause the selected nerve 118 to become habituated to the acoustic wave 114. A habituated nerve can exhibit a decreased interaction with the acoustic wave 114 compared to a non-habituated nerve. However, changing one or more characteristics of the acoustic wave 114 can inhibit nerve habituation by substantially preventing the selected nerve 118 from becoming habituated or reversing the effects of nerve habituation.

In an embodiment, the first or second ultrasound transducer 102, 104 is configured to pulse the first or second ultrasound wave 110, 112, respectively. The first or second ultrasound transducer 102, 104 pulses the first or second ultrasound wave 110, 112, respectively, when the first or second ultrasound transducer 102, 104 controllably and selectively repetitiously increases and decreases the amplitude of the first or second ultrasound wave 110, 112. Decreasing the amplitude of the first or second ultrasound wave 110, 112 can include ceasing to emit the first or second ultrasound wave 110, 112. Each pulse emitted from the first or second ultrasound transducer 102, 104 exhibits a pulse duration measured from a start of increasing the amplitude of one pulse to the start of increasing the amplitude of the next subsequent pulse. In an embodiment, the pulse duration of the first or second ultrasound wave 110, 112 can be on the order of nanoseconds (e.g., about 1 nanosecond to about 1000 nanoseconds), microseconds (e.g., about 1 microsecond to about 1000 microseconds), milliseconds (e.g., about 1 millisecond to about 1000 millisecond), or seconds (e.g., about 1 second to about 60 seconds). Shorter durations (e.g., nanosecond or microsecond range) may be more likely to affect the interaction between the selected nerve 118 and the acoustic wave 114 than longer durations (e.g., microsecond or millisecond range). Meanwhile, longer durations may be more likely to inhibit nerve habituation than shorted durations.

Pulsing the first or second ultrasound wave 110, 112 can cause the acoustic wave 114 to also pulse and exhibit a pulse duration. Pulsing the acoustic wave 114 can change the interaction that the acoustic wave 114 has on the selected nerve 118. For example, pulsing the acoustic wave 114 can cause the acoustic wave 114 to have an excitatory or inhibitory effect on the selected nerve 118 while the non-pulsed acoustic wave 114 may have an opposite effect, no effect, a lesser effect, or a greater effect than the pulsed acoustic wave 114. Further, pulsing the acoustic wave 114 can inhibit nerve habituation.

In an embodiment, the first or second ultrasound transducer 102, 104 is configured to controllably change the pulse duration of the first or second ultrasound wave 110, 112. Changing the pulse duration of the first or second ultrasound wave 110, 112 can change the pulse duration of the acoustic wave 114. Changing the pulse duration of the acoustic wave 114 can change the interaction that the acoustic wave 114 has on the selected nerve 118. Further, changing the pulse duration of the acoustic wave 114 can inhibit nerve habituation.

The first or second ultrasound transducer 102, 104 can be configured to emit the first or second ultrasound wave 110, 112 in a pulse sequence. The pulse sequence, as used herein, refers to pulsing the first or second ultrasound wave 110, 112 in a repetitious pattern. The pulse sequence of the first or second ultrasound wave 110, 112 can cause the acoustic wave 114 to exhibit a pulse sequence. The pulse sequence of the acoustic wave 114 can cause the acoustic wave 114 to interact with the selected never 118 different than if the acoustic wave 114 did not exhibit the pulse sequence. Further, the pulse sequence of the acoustic wave 114 can inhibit nerve habituation. In an embodiment, the pulse sequence includes pulsing the first or second ultrasound wave without changing any characteristics of the first or second ultrasound wave. In an embodiment, the pulse sequence includes pulsing the first or second ultrasound wave 110, 112 while changing one or more characteristics of the first or second ultrasound wave 110, 112. In an embodiment, the pulse sequence can include a first pattern and a second pattern that is different than the first pattern. In an embodiment, the first pattern can include maintaining the characteristics of the first or second ultrasound wave 110, 112 the same while the second pattern can include varying the characteristics of the first or second ultrasound wave 110, 112. In an embodiment, the second pattern can include changing at least one characteristics of the first or second ultrasound wave 110, 112 relative to the first pattern. In an embodiment, the first pattern includes varying the first or second ultrasound wave 110, 112 in a first manner and the second pattern includes varying the first or second ultrasound wave 110, 112 in a second manner that is different than the first pattern. A pulse sequence that includes a first and second pattern can inhibit nerve habituation.

The first ultrasound transducer 102 can be configured to emit the first ultrasound wave 110 in a first pulse sequence and the second ultrasound transducer 104 can be configured to emit the second ultrasound wave 112 in a second pulse sequence. The first and second pulse sequences can be the same or different. The first and second pulse sequence can cause the acoustic wave 114 to exhibit a third pulse sequence. In an embodiment, the first and second pulse sequences are selected to enable the first ultrasound wave 110 and the second ultrasound wave 112 to simultaneously reach the intersection site 116 which allows the first and second ultrasound waves 110, 112 to non-linearly interact with each other. In such an embodiment, the first and second pulse sequence can include emitting the first and second ultrasound waves 110, 112 from the first and second ultrasound transducers 102, 104, respectively, at different times when the first and second ultrasound transducers 102, 104 are spaced at different distances from the intersection site 116. In an embodiment, the first and second pulse sequences are selected to cause the acoustic wave 114 to exhibit a third pulse sequence that has an excitatory or inhibitory effect on the selected nerve 118.

As previously discussed, the nerve modulation device 100 can include the controller 120. The controller 120 can be operably coupled to the first and second ultrasound transducers 102, 104. The controller 120 can be configured to at least partially control the operation of the first and second ultrasound transducers 102, 104. In an embodiment, the controller 120 can be configured to direct the first and second ultrasound transducers 102, 104 to selectively and controllably emit the first and the second ultrasound waves 110, 112, respectively. In an embodiment, the controller 120 can be configured to select a frequency or other characteristics of the first or second ultrasound wave 110, 112. In an embodiment, the controller 120 can be configured to direct the first or second ultrasound transducer 102, 104 to controllably change one or more characteristics of the first or second ultrasound wave 110, 112, respectively. In any of the above embodiment, the controller 120 can at least partially control the operation of the first or second ultrasound transducer 102, 104 by sending one or more directions to the first or second ultrasound transducer 102, 104. The first or second ultrasound transducer 102, 104 can operate in accordance with the directions.

In an embodiment, the controller 120 can include memory 124 (including memory electrical circuitry) storing one or more instructions (e.g., applications, programs, databases, etc.). The one or more instructions can include a pulse sequence, one or more characteristics of the first or second ultrasound waves 110, 112, etc. The controller 120 can also include control electrical circuitry 126 operably coupled to the memory 124. The control electrical circuitry 126 can be configured to execute the one or more instructions stored on the memory 124. For example, the controller 120 can direct at least one of the first or second ultrasound transducer 102, 104 to at least one of emit the first or second ultrasound waves 110, 112, change a frequency of the first or second ultrasound waves 110, 112, pulse the first or second ultrasound waves, etc. responsive to the control electrical circuitry 126 executing the instructions that are stored on the memory 124.

As previously discussed, the nerve modulation device 100 can include at least one sensor 122. The sensor 122 can be communicably coupled to the controller 120. The sensor 122 can be configured to detect one or more characteristics, such as one or more characteristics of the subject 108 or one or more characteristics of the nerve modulation device 100. Responsive to detecting the one or more characteristics, the sensor 122 can transmit one or more sensing signals to the controller 120. The sensing signals can include at least the detected characteristics. In an embodiment, the controller 120 can at least partially direct the operation of the first and second ultrasound transducer 102, 104 responsive to receiving the sensing signals.

The sensor 122 is configured to detect one or more characteristics of the subject 108. In an embodiment, the sensor 122 includes at least one nerve signal sensor configured to detect one or more nerve signals, such as one or more nerve signals that are transmitted or generated at the selected nerve 118. For example, the sensor 122 can be configured to detect one or more pain signals. Responsive to detecting the one or more nerve signals, the sensor 122 can transmit one or more sensing signals to the controller 120. The controller 120 can direct the first and second ultrasound transducers 102, 104 to generated an acoustic wave 114 that is configured to cause an excitatory effect (e.g., if the detected nerve signals indicate a good sensation) or inhibitory effect (e.g., if the detected nerve signals are a negative sensation, such as pain) on the selected nerve 118. In an embodiment, the sensor 122 includes at least one physiological sensor configured to detect one or more physiological characteristics of the subject 108. For example, the physiological sensor can include a blood pressure sensor. In such an embodiment, the blood pressure sensor can transmit one or more sensing signals to the controller 120 that includes the detected blood pressure. The controller 120 can direct the first and second ultrasound transducers 102, 104 to generate an acoustic wave 114 that is configured to cause an excitatory effect on baroreceptors if the detected if the detected blood pressure is high or inhibitory effect on baroreceptors if the detected blood pressure is low. In another example, the physiological sensor can include a heart rate sensor, a temperature sensor, an oximeter, an electrophysiological sensor, skin conductance sensor, a bioimpedance sensor, a chemical sensor, a pH sensor, etc. In an embodiment, the sensor 122 can include at least one sensor configured to detect tremors, such as an image sensor (e.g., a charge-coupled device, a complementary metal-oxide-semiconductor sensor), an optical sensor (e.g., oximeter or position sensor), an accelerometer, or an electromyograph (e.g., surface electromyograph). In such an embodiment, the controller 120 can direct the first and second ultrasound transducers 102, 104 to generate an acoustic wave 114 configured to modulate a nerve that is causing or can regulate the tremors. Depending on the cause of the tremors, the acoustic wave 114 can be configured to have an excitatory or inhibitory effect on the nerve.

The sensor 122 can be configured to detect one or more characteristics of the nerve modulation device 100. In an embodiment, the sensor 122 is configured to detect one or more characteristics of the first or second ultrasound transducer 102, 104. For example, the sensor 122 (e.g., a piezoelectric or other ultrasound sensor) can be configured to detect one or more characteristics (e.g., frequency, amplitude, directionality, pulse duration, pulse sequence) of the first or second ultrasound wave 110, 112. In such an embodiment, the controller 120 can direct the first or second ultrasound transducer 102, 104 to change one or more characteristics of the first or second ultrasound wave 110, 112. In an embodiment, the sensor 122 is configured to detect one or more characteristics of the acoustic wave 114. For example, the sensor 122 (e.g., a piezoelectric or other sound sensor) can be configured to detect one or more characteristics of the acoustic wave 114. In such an embodiment, the sensor 122 can transmit one or more sensing signals to the controller 120 and the controller 120 can direct at least one of the first or second ultrasound transducers 102, 104 to change one or more characteristics of the first or second ultrasound wave 110, 112 thereby modifying the acoustic wave 114.

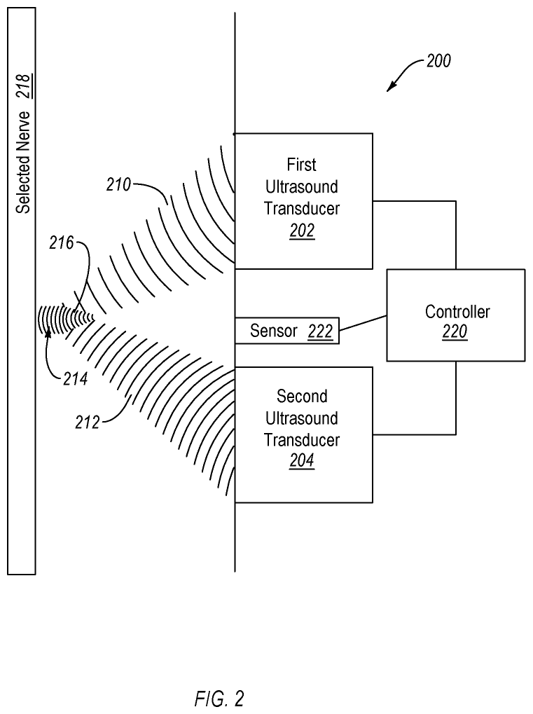

As previously discussed, the nerve modulation device 100 is configured such that the intersection site 116 of the first and second ultrasound waves 110, 112 is at the selected nerve 118. However, the intersection site 116 of the first and second ultrasound waves 110, 112 can be spaced from the selected nerve 118. For example, FIG. 2 is a schematic view of a nerve modulation device 200 that is configured to have an intersection site 216 of a first ultrasound wave 210 and second ultrasound wave 212 that is near (e.g., spaced from and proximate to) a selected nerve 218, according to an embodiment. Except as otherwise disclosed herein, the nerve modulation device 200 is the same as or substantially similar to any of the nerve modulation devices disclosed herein. For example, the nerve modulation device 200 includes a first ultrasound transducer 202 that is configured to emit the first ultrasound wave 210 and a second ultrasound transducer 204 that is configured to emit the second ultrasound wave 212. The nerve modulation device 200 can also include a controller 220 and at least one sensor 222.

The first ultrasound transducer 202 is configured to emit the first ultrasound wave 210 in a first direction and the second ultrasound transducer 204 is configured to emit the first ultrasound wave 212 in a second direction. The first and second directions are selected such that the first and second ultrasound waves 210, 212 intersect at an intersection site 216 that is selected to be near the selected nerve 218. The intersection site 216 is selected to be proximate to the selected nerve 218, which can facilitate the acoustic wave 214 remaining relatively focused when the acoustic wave 214 reaches the selected nerve 218. In such an embodiment, the intersection site 216 can be about 0.25 mm to about 0.75 mm, about 0.5 mm to about 1 mm, about 0.75 mm to about 1.5mm, about 1 mm to about 2 mm, about 1.5 mm to about 3 mm, or about 2.5 mm to about 5 mm from the selected nerve 218.

The first and second ultrasound waves 210, 212 non-linearly interact at the intersection site 216 to form an acoustic wave 214. The acoustic wave 214 can be emitted from the intersection site 216 in a third direction. The third direction is selected generally extend from the intersection site 216 towards the selected nerve 218. The third direction can be different than at least one (e.g., both) of the first and second directions of the first and second ultrasound waves 210, 212. The third direction of the acoustic wave 214 can depend on a number of factors, such as at least one of the first frequency of the first ultrasound wave 210, the second frequency of the second ultrasound wave 212, the first direction of the first ultrasound wave 210, or the second direction of the second ultrasound wave 212.

Selecting the intersection site 216 to be near the selected nerve 218 can prevent or limit the amount of the first and second ultrasound waves 210, 212 that reach the selected nerve 218. As such, selecting the intersection site 216 to be near (but not at) the selected nerve 218 can prevent nerve damage and interference with interaction between the acoustic wave 214 and the selected nerve 218.

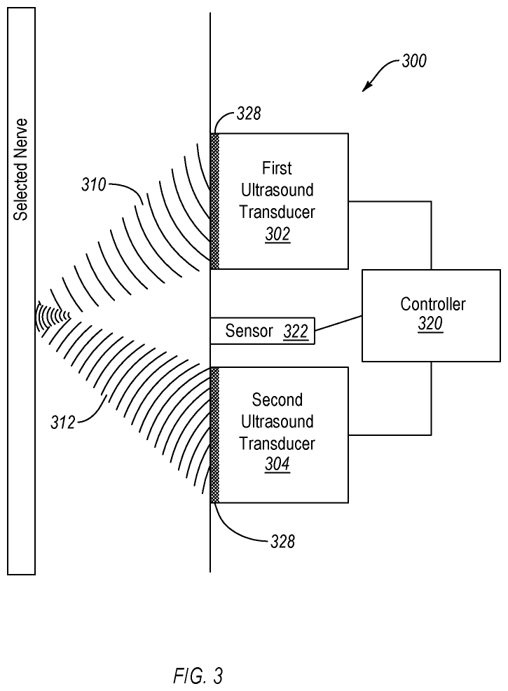

FIG. 3 is a schematic view of a nerve modulation device 300, according to an embodiment. Except as otherwise disclosed herein, the nerve modulation device 300 is the same as or substantially similar to any of the nerve modulation devices disclosed herein. For example, the nerve modulation device 300 includes a first ultrasound transducer 302 that is configured to emit the first ultrasound wave 310 and a second ultrasound transducer 304 that is configured to emit the second ultrasound wave 312. The nerve modulation device 300 can also include a controller 320 and at least one sensor 322.

The nerve modulation device 300 includes a ultrasound-conductive material 328. The ultrasound-conductive material 328 can be disposed between the first or second ultrasound transducer 302, 304 and an external surface 306 of a subject 308. The ultrasound-conductive material 328 can be configured to substantially completely fill the space between the first or second ultrasound transducer 302, 304 and the external surface 306 since ultrasound waves typically do not travel well through air. Further, the ultrasound-conductive material 328 can form a tight bond between the first or second ultrasound transducers 302, 304 and the external surface 306 thereby allowing the first or second ultrasound waves 310, 312 to travel directly into the subject 308. In an embodiment, the ultrasound-conductive material 328 can include an ultrasound gel, such as a gel including at least one of propylene glycol, glycerine, or phenoxyethanol. In an embodiment, the ultrasound-conductive material 328 can include guar gum. In an embodiment, the ultrasound-conductive material 328 can include water.

The nerve modulation devices 100, 200, 300 of FIGS. 1-3 include ultrasound transducers that are positioned on an external surface of a subject. The ultrasound transducers can be coupled to and maintained on the external surface of the subject using any suitable method, such as by manually holding the ultrasound transducers in place. In an embodiment, the ultrasound transducers can coupled to and maintained on the external surface using at least one attachment device. The attachment device can include any suitable device that is configured to provide a force to at least one of the ultrasound transducers directing the ultrasound transducer towards the external surface of the subject. The force applied by the attachment device can include a pushing force (e.g., a strap) or a pushing force (e.g., a suction cup). The force applied by the attachment device can improve contact between the ultrasound transducer and the external surface (e.g., minimizing the amount of air between the ultrasound transducer and the external surface) thereby facilitating efficient operation of the nerve modulation device. In an embodiment, the at least one attachment device can include a single attachment device that couples each non-implanted ultrasound transducer to the subject or two or more attachment devices that each couples at least one of the non-implanted ultrasound transducers to the subject. In an embodiment, the attachment devices disclosed herein can support one or more additional components of the nerve modulation device, such as the sensor or the controller.

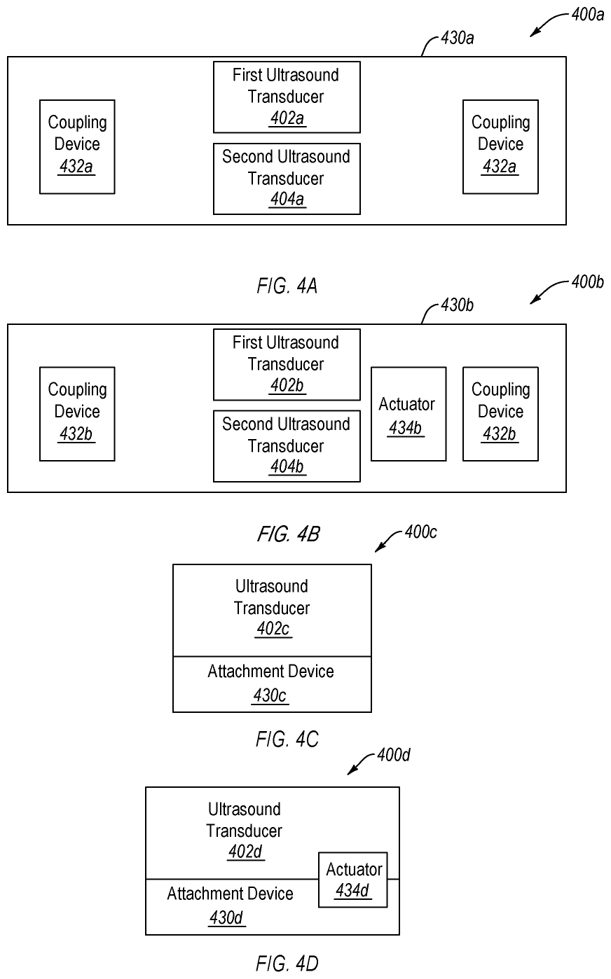

FIGS. 4A-4D are schematics of at least a portion of different nerve modulation devices that each include at least one attachment device configured to couple the ultrasound transducers of the different nerve modulation devices to the external surface of the subject, according to different embodiments. Except as otherwise disclosed herein, the nerve modulation devices shown in FIGS. 4A-4D are the same as or substantially similar to any of the nerve modulation devices disclosed herein. Similar, the attachment devices illustrated in FIGS. 4A-4D can be used in any of the embodiments disclosed herein.

FIG. 4A illustrates a nerve modulation device 400a that includes at least one attachment device 430a. The attachment device 430a is a wearable apparatus that is configured to be worn by the subject. The attachment device 430a can be configured to hold one or more ultrasound transducers and to apply a force to the ultrasound transducers that directs the ultrasound transducers towards the external surface of the subject. For example, the nerve modulation device 400a can includes a first ultrasound transducer 402a and a second ultrasound transducer 404a. At least one of the first or second ultrasound transducer 402a, 404a can be coupled to the attachment device 430a such that the attachment device 430a holds and supports the first or second ultrasound transducer 402a, 404a.

The attachment device 430a can include any wearable apparatus. In an embodiment, the attachment device 430a can include a band (e.g., a wrist band), a compression garment, a portion of a larger garment (e.g., a pocket in a garment), or any other suitable wearable apparatus. In an embodiment, the attachment device 430a can include a wearable apparatus that is skin tight such that merely wearing the attachment device 430a applies a force (e.g., a pushing force) against the ultrasound transducers coupled thereto directing the ultrasound transducers against the external surface of the subject. In such an embodiment, the attachment device 430a can be formed from a fabric or material that stretches which can allow the attachment device 430a to be used with different geometries of the external surface and can make the attachment device 430a more comfortable to wear compared to other fabric or material. In an embodiment, the attachment device 430a can include or form part of a wearable apparatus that is loose (e.g., not skin tight). In such an embodiment, the attachment device 430a can include one or more components that pull the ultrasound transducers coupled thereto against the external surface of the subject. The one or more components can include straps, bands, etc. The one or more components can be formed for a fabric or material that stretches which can allow the attachment device 430a to be used with different geometries of the external surface or make the attachment device 430a more comfortable to wear.

The attachment device 430a can be configured to be reversibly coupled to the subject. In an embodiment, the attachment device 430a can be flexible or elastic thereby allowing the attachment device 430a to be easily coupled to and removed from the subject. In an embodiment, the attachment device 430a can include a coupling device 432a. The coupling device 432a can include Velcro, at least one button, at least one snap, a zipper, a clamp, a pin, or any other suitable device. The coupling device 432a can be configured to switch the attachment device 430a between a closed state and an open state. For example, the attachment device 430a can exhibit a small circumference or a restrictive shape when the attachment device 430a is in the closed state. The attachment device 430a can exhibit the small circumference or restrictive shape when portions of the coupling device 432a are coupled together or coupled to another portion of the attachment device 430a. The small circumference or restrictive shape of the attachment device 430a can be selected to stably secure the attachment device 430a to the subject. The coupling device 432a can switch the attachment device 430a from the closed state to the open state by decoupling the portions of the coupling device 432a from each other or from the attachment device 430a. Decoupling the portions of the coupling device 432a can increase the circumference of the attachment device 430a or allow the shape of the attachment device 430a to change (e.g., from an annular shape to a strip-like shape). Increasing the circumference of the attachment device 430a or allowing the shape of the attachment device 430a to change can allow the attachment device 430a to be unsecured to the subject and facilitate attaching and removing the attachment device 430a from the subject.

FIG. 4B illustrates a nerve modulation device 400b, according to an embodiment. Except as otherwise disclosed herein, the nerve modulation device 400b can be the same as or similar to the nerve modulation device 400a of FIG. 4A. For example, the nerve modulation device 400b can include an attachment device 430b, a first or second ultrasound transducer 402b, 404b, and a coupling device 432b.

The nerve modulation device 400b can include an actuator 434b (e.g., electric motor, pneumatic or hydraulic actuator, a shape memory alloy, etc.) that is configured to switch the nerve modulation device 400b between a relatively tight state and a relatively loose state. For example, as previously discussed, the attachment device 430b can apply a force the ultrasound transducers coupled thereto that directs the ultrasound transducers towards the external surface of the subject. The force applied to the ultrasound transducers improves the contact between the ultrasound transducer and the subject which facilitates efficient operation of the nerve modulation device 400b. When the nerve modulation device 400b is in the tight state, the attachment device 430b can apply a first force to the ultrasound transducers that are coupled thereto. The first force can be sufficient to press the ultrasound transducers that are coupled to the attachment device 430a against and, optionally, into the subject thereby improving contact between the ultrasound transducers and the subject. However, the nerve modulation device 400b can be uncomfortable to wear when the nerve modulation device 400b is in the tight state because the nerve modulation device 400b may decrease blood circulation, inhibit movement, press the ultrasound transducers into the subject, etc. As such, the actuator 434b can be configured to switch the nerve modulation device 400b from the tight state to a loose state by increasing the circumference of the attachment device 430b or allowing the shape of the attachment device 430b to change. However, unlike the open state of the attachment device 430b, the actuator 434b switches the state of the nerve modulation device 400b instead of the coupling device 432b and the nerve modulation device 400b may remain stably or substantially stably secured to the subject while the nerve modulation device 400b is in the loose state. The attachment device 430b applies a second force to the ultrasound transducers coupled thereto that is less than the first force when the nerve modulation device 400b is in the loose state. The second force applied to the ultrasound transducers can make the nerve modulation device 400b more comfortable to wear. However, the contact between the ultrasound transducers that are coupled to the attachment device 430b and the external surface can be worse when the nerve modulation device 400b is in the loose state than when the nerve modulation device 400b is in the tight state. As such, the nerve modulation device 400b may only exhibit the loose state when the nerve modulation device 400b (e.g., the first and second ultrasound transducers 402b, 404b) is not emitting ultrasound waves into the subject while the nerve modulation device 400b may only exhibit the tight state when the nerve modulation device 500b is emitting ultrasound waves.

FIG. 4C is a schematic of a portion of a nerve modulation device 400c, according to an embodiment. The nerve modulation device 400c includes an ultrasound transducer 402c and an attachment device 430c coupled to the ultrasound transducer 402c. The attachment device 430c is configured to apply a force (e.g., pulling force) to the ultrasound transducer 402c. In an embodiment, the attachment device 430c can include a suction cup that is configured to be coupled to an external surface of the subject. Coupling the suction cup to the external surface of the subject can cause the attachment device 430c to pull the ultrasound transducers 402c against the external surface. In an embodiment, the attachment device 430c can include an adhesive, tape (e.g., double sided tape), or any other suitable attachment device.

In an embodiment, the attachment device 430c is only coupled to the ultrasound transducer 402c. In an embodiment, the attachment device 430c is coupled to the ultrasound transducer 402c and one or more additional components (not shown) of the nerve modulation device 400c, such as at least one additional ultrasound transducer, a controller, or at least one sensor.

FIG. 4D is a schematic of a portion of a nerve modulation device 400d, according to an embodiment. Except as otherwise disclosed herein, the nerve modulation device 400d can be the same as or substantially similar to the nerve modulation device 400c of FIG. 4C. For example, the nerve modulation device 400d can include an ultrasound transducer 402d and an attachment device 430d coupled to the ultrasound transducer 402d.

The nerve modulation device 400d can include an actuator 434d that is configured to switch the nerve modulation device 400d between a tight state and a loose state. As previously discussed, the attachment device 430d can apply a first force to the ultrasound transducer 402 when the nerve modulation device 400d is in the tight state (e.g., when the ultrasound transducer emits ultrasound waves). However, the first force can make the nerve modulation device 400d uncomfortable to wear. For example, when the attachment device 430d is a suction cup or similar device, the first force can be a suction force that is sufficient to cause blood vessels at or near the external surface of the subject to rupture. As such, the actuator 434d can switch the nerve modulation device 400d from the tight state to the loose state wherein the attachment device 430d applies a second force that is less than the first force to the ultrasound transducer 402d when the nerve modulation device 400d is in the loose state. The second force can make the nerve modulation device 400d more comfortable to wear. The actuator 434d can include any actuator that is configured to switch the nerve modulation device 400d from the tight state to the loose state, such as an vacuum pump or any other actuator disclosed herein.

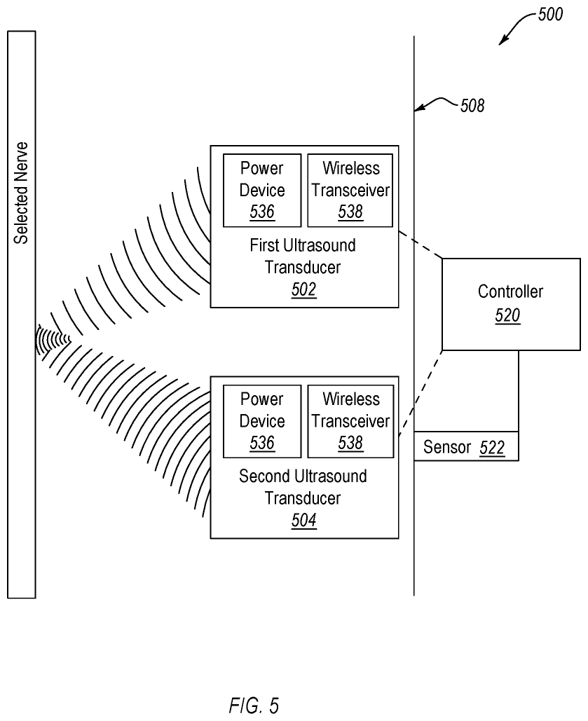

The nerve modulation devices illustrated in FIGS. 1-3 are illustrated as being at least partially positioned on an external surface of the subject. However, one or more components of the nerve modulation devices disclosed herein can be implanted in the subject. For example, FIG. 5 is a schematic view of a nerve modulation device 500 that includes one or more components implanted in the subject 508, according to an embodiment. For instance, the one or more components implanted in the subject 508 can be implanted via surgery, a needle, or any other suitable method. While implanting one or more components of the nerve modulation device 500 in the subject 508 can be intrusive, implanting one or more components in the subject 508 can be beneficial when the nerve modulation device 500 will be used for a prolonged period of time (e.g., at least a day, at least a week, at least a month, or at least a year). Additionally, implanting the one or more components of the nerve modulation device 500 in the subject 508 can prevent the components from inadvertently moving relative to the subject 508 during operation.

Except as otherwise disclosed herein, the nerve modulation device 500 can be the same as or substantially similar to any of the nerve modulation devices disclosed herein. For example, the nerve modulation device 500 can include a first ultrasound transducer 502, a second ultrasound transducer 504, a controller 520, and at least one sensor 522.

As previously discussed, one or more components of the nerve modulation device 500 can be implanted in the subject. For example, as illustrated, the first and second ultrasound transducers 502, 504 can be implanted in the subject 508 and the controller 520 and the sensor 522 are disposed outside of the subject 508. However, it is noted that at least one of the first and second ultrasound transducers 502, 504 can be disposed outside of the subject 508, the controller 520 can be implanted in the subject 508, or the sensor 522 can be implanted in the subject 508.

In an embodiment, the components of the nerve modulation device 500 that are configured to be implanted can be waterproof or water resistant since the components may be exposed to bodily fluids. In an embodiment, the components of the nerve modulation device 500 that are configured to be implanted can be formed from a biocompatible material. In an embodiment, the components of the nerve modulation device 500 that are configured to be implanted can include a power source 536 that is configured to power the component. The power source 536 can include a battery or any other suitable power device. In an embodiment, the power source 536 can include a wireless charger configured to wireless receive electrical power from a source that is dispose outside of the subject 508. The wireless charger can be electrical coupled to and configured to supply power to a rechargeable battery or any other device of the component. The wireless charger can include, for example, an inductive charging device, capacitive coupling device, RFIDs, or any other suitable device. In an embodiment, the components of the nerve modulation device 500 that are configured to be implanted can include a wireless transceiver 538 that is configured to communicably coupled the implanted components to devices that are spaced therefrom, such as components that are not implanted. Examples of the wireless transceiver 538 include a Wi-Fi device or a Bluetooth device.

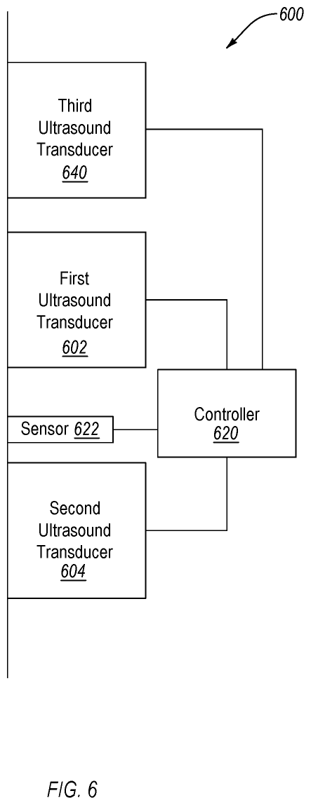

The nerve modulation devices disclosed herein can include three or more ultrasound transducers. For example, FIG. 6 is a schematic view of a nerve modulation device 600 that includes three ultrasound transducers, according to an embodiment. Except as otherwise disclosed herein, the nerve modulation device 600 can be the same as or substantially similar to any of the nerve modulation devices disclosed herein. For example, the nerve modulation device 600 can include a first ultrasound transducer 602 configured to emit a first ultrasound wave exhibiting a first frequency, a second ultrasound transducer 604 configured to emit a second ultrasound wave exhibiting a second frequency, a controller 620, and at least one sensor 622.

The nerve modulation device 600 also includes a third ultrasound transducer 640 that is the same as or substantially similar to any of the ultrasound transducers disclosed herein. For example, the third ultrasound transducer 640 can be configured to emit a third ultrasound wave exhibiting a third frequency in a third direction. The third frequency can be different than at least one of the first frequency of the first ultrasound wave, the second frequency of the second ultrasound wave, or a frequency of an acoustic wave formed by non-linearly interacting the first and second ultrasound waves. The third direction can be the same as or different than at least one of the first direction of the first ultrasound wave or the second direction of the second ultrasound wave. It is noted that the nerve modulation device 600 can include one or more additional ultrasound transducers that are configured to emit ultrasound waves. The frequency and direction of the ultrasound waves emitted from the additional ultrasound transducers can be the same as or different than at least one of the first ultrasound wave, the second ultrasound wave, the third ultrasound wave, or any acoustic wave formed by non-linearly interacting at least two of the first, second, or third ultrasound waves.

In an embodiment, the third frequency of the third ultrasound wave can be different than the first frequency of the first ultrasound wave and the second frequency of the second ultrasound wave. In such an embodiment, the third direction can be selected such that the first, second, and third ultrasound waves intersect with each other at an intersection site that is at or near a selected nerve. The first, second, and third ultrasound waves can non-linearly interact with each other to form an acoustic wave. The acoustic wave (e.g., a frequency or directionality of the acoustic wave) may be difficult or impossible to form by non-linearly interacting only the first and second ultrasound waves together. In an embodiment, the third frequency of the third ultrasound wave can be different than the first frequency of the first ultrasound wave. In such an embodiment, the third direction can be selected such that the first and third ultrasound waves intersect at a first intersection site to form a first acoustic wave. The frequency of the first acoustic wave can be the same as or similar to any of the acoustic frequencies or ultrasound frequencies disclosed herein. The first acoustic wave may be emitted from the first intersection site in a general direction that allows the first acoustic wave to intersect the second ultrasound wave at a second intersection site that can be closer to a selected nerve than the first intersection site. The frequency of the first acoustic wave can be different than the second frequency of the second ultrasound wave and, as such, the first acoustic wave and the second ultrasound wave can non-linearly interact to form a second acoustic wave. The second acoustic wave may be difficult or impossible to form by non-linearly interacting only the first and second ultrasound waves together. In an embodiment, the third frequency of the third ultrasound wave can be the same as the first frequency of the first ultrasound wave. In such an embodiment, the third direction of the third ultrasound wave can be selected to intersect the first and third ultrasound waves. Intersecting the first and third ultrasound waves can cause the third ultrasound wave to constructively or destructively interfere with the first ultrasound wave thereby increasing or decreasing an amplitude of the first ultrasound wave. In an embodiment, the third frequency of the third ultrasound wave can be the same as a frequency of an acoustic wave formed by non-linearly interacting the first and second ultrasound waves. In such an embodiment, the third direction of the third ultrasound wave can be selected to intersect the acoustic wave. Intersecting the third ultrasound wave with the acoustic wave can cause the third ultrasound wave to constructively or destructively interfere with the acoustic wave thereby increasing or decreasing an amplitude of the acoustic wave. In an embodiment, the third direction of the third ultrasound wave can be selected such that the third ultrasound wave does not intersect the first ultrasound wave, the second ultrasound wave, and an acoustic wave formed by non-linearly interacting the first ultrasound wave with the second ultrasound wave. In such an embodiment, for example, the third ultrasound wave can interact with a different portion of the same nerve as the acoustic wave, interact with a nerve that is different than the nerve that is modulated by the acoustic wave, or the third ultrasound wave can intersect with and non-linearly interact with an additional ultrasound emitted from an addition ultrasound transducer.

In an embodiment, any of the ultrasound transducers disclosed herein can be configured to controllably change the directions that the ultrasound transducers emit ultrasound waves therefrom. For example, the ultrasound transducers can change the directions that the ultrasound transducers emit ultrasound waves because of, for example, a different portion of a nerve or a different nerve is going to be modulated, the directionality of an ultrasound wave is incorrect to form an acoustic wave, the ultrasound transducer is inadvertently moved, etc.

The ultrasound transducers can change the direction that the ultrasound transducers emit the ultrasound waves responsive to direction for the controller. In an embodiment, the controller can direct the ultrasound transducer to change the direction that the ultrasound transducers emits the ultrasound waves responsive to receiving one or more sensing signals from at least one sensor. In an embodiment, the sensing signals can include one or more characteristics of the ultrasound transducer (e.g., of the ultrasound waves emitted from the ultrasound transducer) that is detected by the sensor, such as that the direction that the ultrasound wave is emitted is incorrect, the ultrasound transducer is incorrectly positioned, or the ultrasound transducer was moved. In an embodiment, the sensing signals can include one or more characteristics of an acoustic wave. The characteristics of the acoustic wave can include whether or not the acoustic wave is formed or whether the acoustic wave is interacting with the selected nerve. In an embodiment, the sensing signals can include one or more physiological characteristics of the subject, such as whether the subject is experiencing a desired effect from the neural modulation. In an embodiment, the controller can direct the ultrasound transducer to change the direction that the ultrasound transducer emits the ultrasound waves responsive to input from the subject or another individual (e.g., nurse or medical practitioner). The input from the subject or another individual can include whether or not the nerve modulation is creating the desired effect.

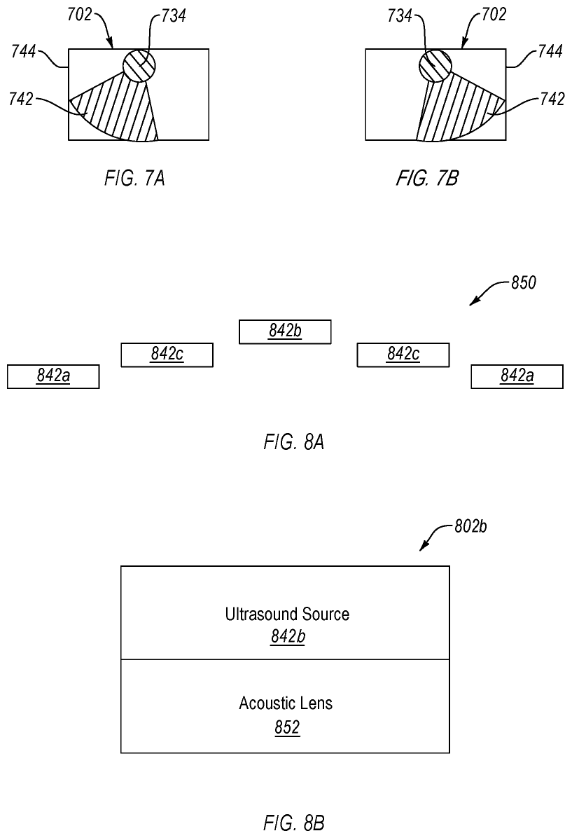

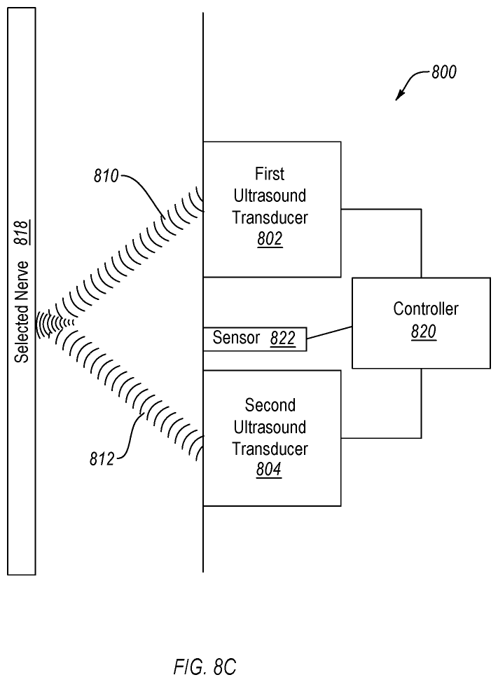

The ultrasound transducers can change the direction that the ultrasound transducers emit ultrasound waves using any suitable method. FIGS. 7A and 7B are schematic cross-sectional views of an ultrasound transducer 702 that is configured change a direction that the ultrasound transducer 702 emits ultrasound waves, according to an embodiment. The ultrasound transducer 702 can include an ultrasound source 742, a housing 744, and an actuator 734 (e.g., any of the actuators disclosed herein) that moveably couples the ultrasound source 742 to the housing 744. Referring to FIG. 7A, the ultrasound source 742 can exhibit a first position relative to the housing 744. The ultrasound source 742 can emit ultrasound waves in a first direction relative to the housing 744 when the ultrasound source 742 is in the first position. The actuator 734 can receive direction from a controller (not shown) directing the actuator 734 to change the position of the ultrasound source 742 relative to the housing 744. Responsive to receiving the direction from the controller, as shown in FIG. 7B, the actuator 734 can move the ultrasound source 742 to a second position relative to the housing 744. The ultrasound source 742 can emit ultrasound waves in a second direction relative to the housing 744 when the ultrasound source 742 is in the second position. Since the housing 744 can remain stationary relative to a subject, moving the ultrasound source 742 from the first position to the second position can change the direction that the ultrasound source 742 emits the ultrasound waves. It is noted that the ultrasound transducers disclosed herein can change the direction that ultrasound wave are emitted therefrom using other methods, such as discussed in more detail with regards to FIG. 8B.

As previously discussed herein, the ultrasound transducers disclosed herein can emit focused ultrasound waves. FIGS. 8A and 8B illustrate different methods of focusing an ultrasound wave, though other methods can be used. FIG. 8A is a schematic illustration of an ultrasound array 850 that can be used in any of the ultrasound transducers disclosed herein, according to an embodiment. The ultrasound array 850 includes a plurality of ultrasound sources, such as at least two outermost ultrasound sources 842a, at least one centermost ultrasound source 842b, and, optionally, at least one intermediate ultrasound source 842c disposed between the outermost ultrasound sources 842a and the centermost ultrasound source 842b. The ultrasound sources can be formed in a concave curvature in which each of the ultrasound sources emit distinct ultrasound waves in the general direction that the concave curvature faces or can be formed in a linear or planar pattern. The ultrasound sources of the ultrasound array 850 are each configured to emit distinct ultrasound waves that, collectively, form a focused ultrasound array. The ultrasound array 850 forms the focused ultrasound array by controllably emitting the distinct ultrasound waves from the ultrasound sources at different times. In particular, the ultrasound sources that are further spaced from the centermost ultrasound source 842b emits the distinct ultrasound wave therefrom before the ultrasound sources that are closer to the centermost ultrasound source 842b emits the distinct ultrasound wave therefrom. For example, the outermost ultrasound sources 842a emit the ultrasound waves therefrom at a first time, the intermediate ultrasound sources 842c emit the ultrasound waves therefrom at a second time that is after the first time, and the centermost ultrasound source 842b emits the distinct ultrasound wave therefrom at a third time that is after the first and second times.

It is noted that the ultrasound array 850 can controllably change the direction that the ultrasound wave (e.g., the ultrasound wave formed from the distinct ultrasound waves) emitted therefrom without moving the ultrasound array 850 or the ultrasound sources. For example, the ultrasound array 850 can change the direction that the ultrasound wave is emitted therefrom by emitting the distinct ultrasound waves from ultrasound sources on one side of the centermost ultrasound source 842b before the ultrasound sources on the opposing side of the centermost ultrasound source 842b.

FIG. 8B is a schematic view of an ultrasound transducers 802b, according to an embodiment. Except as otherwise disclosed herein, the ultrasound transducer 802b can be the same as or substantially similar to any of the ultrasound transducers disclosed herein. For example, the ultrasound transducer 802b can include an ultrasound source 842. However, the ultrasound transducer 802b includes an acoustic lens 852 that is configured to focus ultrasound waves emitted from the ultrasound source 842. The acoustic lens 852 can include any suitable acoustic lens, such as at least one hyperbolic plate or a collection of perforated barriers. The ultrasound transducer 802b can used in any of the ultrasound transducers disclosed herein.

In an embodiment, the ultrasound transducers disclosed herein may not emit focused ultrasound waves. For example, FIG. 8C is a schematic view of a nerve modulation device 800c that is configured to emit at least one unfocused and uniform ultrasound wave, according to an embodiment. Except as otherwise disclosed herein, the nerve modulation device 800c can be the same as or substantially similar to any of the nerve modulation devices disclosed herein. For example, the nerve modulation device 800 can include a first ultrasound transducer 802, a second ultrasound transducer 804, a controller 820, and at least one sensor 822. The first and second ultrasound transducers 802, 804 are configured to emit first and second ultrasound waves 810, 812 exhibiting first and second frequencies, respectively. Each of the first and second ultrasound waves 810, 812 can exhibit a Fresnel length and the first and second ultrasound waves 810, 812 may not substantially converge or diverge when the first and second ultrasound waves 810, 812 are spaced from the first and second ultrasound transducers 802, 804 that is less than the Fresnel length. The Fresnel length of the first and second ultrasound waves 810, 812 is related to the diameter “D” and the wavelength “λ” thereof by the equation Fresnel length=D²/(4λ). When the distance between at least one of the first or second ultrasound transducers 802, 804 to a selected nerve 818 is less than the Fresnel length of the corresponding one of the first or second ultrasound waves 810, 812, the corresponding one of the first or second ultrasound waves 810, 812 can be unfocused. In an embodiment, it is noted that the first or second ultrasound waves 810, 812 can still be focused even when a corresponding one of the first or second ultrasound transducers 802, 804 is spaced from the selected nerve 818 by a distance that is less than the Fresnel length, for example, when the diameter of the first or second ultrasound wave 810, 812 is too large or more control over the first or second ultrasound wave 810, 812 is desired.

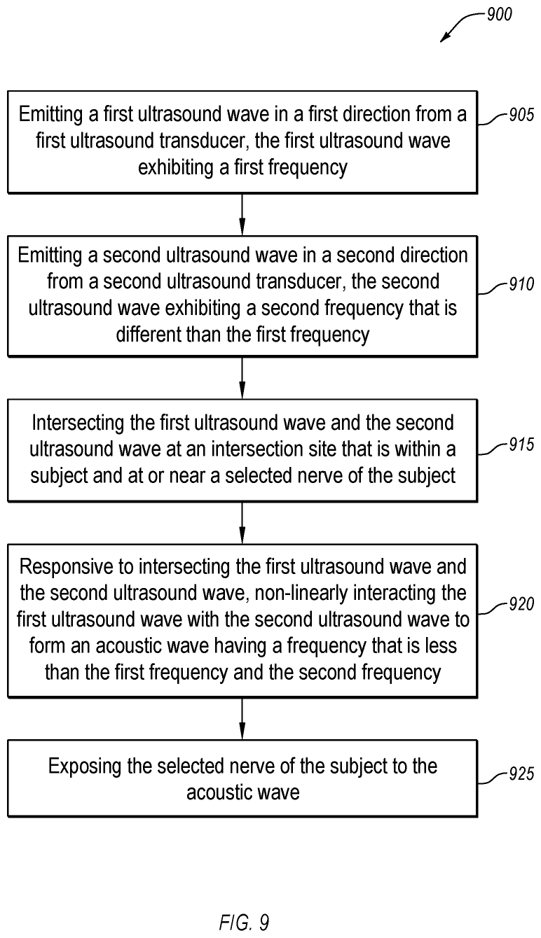

FIG. 9 is a flow diagram of a method 900 of using any of nerve modulation devices disclosed herein, according to an embodiment. In some embodiments, some of the acts of the method 900 can be split into a plurality of acts, some of the acts can be combined into a single act, some acts can be omitted, or some of the acts can be performed in a different order than shown in FIG. 9. Also, it is understood that additional acts can be added to the method 900.

The method 900 can include act 905, which recites “emitting a first ultrasound wave in a first direction from a first ultrasound transducer, the first ultrasound wave exhibiting a first frequency.” Act 905 may be followed by or performed substantially simultaneously with act 910, which recites “emitting a second ultrasound wave in a second direction from a second ultrasound transducer, the second ultrasound wave exhibiting a second frequency that is different than the first frequency.” Responsive to acts 905 and 910, the method 900 can include act 915, which recites “intersecting the first ultrasound wave and the second ultrasound wave at an intersection site that is within the subject and at or near a selected nerve.” Act 915 may be followed by or performed substantially simultaneously with act 920, which recites “responsive to intersecting the first ultrasound wave and the second ultrasound wave, non-linearly interacting the first ultrasound wave with the second ultrasound wave to form an acoustic wave having a frequency that is less than the first frequency and the second frequency.” Act 920 may be followed by or performed substantially simultaneously with act 925, which recites “exposing the selected nerve of the subject to the acoustic wave.”

In an embodiment, act 905 or 910 includes emitting the first or second ultrasound wave from the first or second ultrasound transducer that is positioned on the subject. In an embodiment, act 905 or 910 includes emitting the first or second ultrasound wave from the first or second ultrasound transducer that is implanted in the subject. In an embodiment, act 905 or 910 includes focusing the first or second ultrasound wave towards the intersection site. In such an embodiment, focusing the first or second ultrasound wave can include focusing the first or second ultrasound wave using an ultrasound array by emitting a plurality of distinct ultrasound waves from a plurality of ultrasound source at different times or using an acoustic lens. In an embodiment, act 905 or 910 includes emitting the first or second ultrasound wave in an unfocused and uniform manner towards the intersection site. In such an embodiment, the first or second ultrasound transducer is positioned from the intersection site by a distance that is less than the Fresnel length of the first or second ultrasound wave.

In an embodiment, acts 905 and 910 includes pulsing the first ultrasound wave and the second ultrasound wave. In such an embodiment, pulsing the first and second ultrasound waves can include pulsing the first ultrasound wave at a first pulse sequence and pulsing the second ultrasound wave at a second pulse sequence. In an embodiment, the first pulse sequence and the second pulse sequence are configured to enable the first ultrasound wave and the second ultrasound wave to simultaneously reach the intersection site. In an embodiment, the first pulse sequence and the second pulse sequence are configured to cause the acoustic wave to exhibit a third pulse sequence. In such an embodiment, the third pulse sequence is configured to provide an excitatory or inhibitory effect on the selected nerve.

In an embodiment, act 915 of intersecting the first ultrasound wave and the second ultrasound waves includes intersecting the first and second ultrasound waves at an intersection site that is at the selected nerve. In an embodiment, act 915 of intersecting the first ultrasound wave and the second ultrasound wave includes intersecting the first and second ultrasound waves at an intersection site that is near (e.g., spaced from and proximate to) the selected nerve. In such an embodiment, the method 900 can include emitting the acoustic wave from the intersection site in a third direction that generally extends from the intersection site towards the selected nerve, wherein the intersection site is near the selected nerve.

Act 925 can include exposing baroreceptors, nerves in the neck, or any other suitable nerve to the acoustic wave. Exposing the selected nerve to the acoustic wave can include providing an excitatory or inhibitory effect to the selected nerve.

The method 900 can include, before acts 905 and 910, positioning the first or second ultrasound transducer against an external surface of the subject and maintaining the first ultrasound transducer against the external surface using at least one attachment device. The attachment device can provide a force directing the first or second ultrasound transducer towards the external surface of the subject. In an embodiment, the method 900 can include controllably switching the nerve modulation device between a tight state and a loose state with at least one actuator coupled to the at least one attachment device. In such an embodiment, the force provided by the at least one attachment device is greater when the nerve modulation device is in the tight state than when the nerve modulation is in the loose state.

In an embodiment, positioning the first or second ultrasound transducer can include positioning an ultrasound-conductive material between the first or second ultrasound transducer and the external surface of the subject. In an embodiment, positioning the first or second ultrasound transducer can include positioning the first or second ultrasound transducer to emit the first or second ultrasound wave, respectively, at or near the selected nerve of the subject. In an embodiment, positioning the first or second ultrasound transducer can include positioning the first ultrasound transducer on a neck of the subject since many of the nerves that can be modulated by the nerve modulation device may be located at or near the neck of the subject.

The method 900 can include detecting one or more characteristics with at least one sensor. Detecting one or more characteristics with the sensor can include detecting one or more characteristics of at least one of the subject, the first ultrasound transducer, the second ultrasound transducer, the first ultrasound wave, the second ultrasound wave, or the acoustic wave. Detecting one or more characteristics of the subject can include detecting one or more nerve signals with a nerve signal sensor or detecting one or more physiological characteristics of the subject with a physiological sensor. Examples of detecting one or more physiological characteristics of the subject with the physiological sensor can include detecting tremors or lack of tremors in the subject with an image sensor, an optical sensor, or any other suitable sensor or detecting blood pressure of the subject with an oximeter or other suitable sensor. Responsive to detecting the one or more characteristics, the method 900 can include transmitting one or more sensing signals from the at least one sensor and receiving the one or more sensing signals at a controller.

In an embodiment, responsive to receiving the one or more sensing signals at a controller, the method 900 can include controlling at least one operation of the first ultrasound transducer or the second ultrasound transducer with the controller. In an embodiment, controlling at least one operation of the first or second ultrasound transducer with the controller includes directing the first and second ultrasound transducers to emit the first and second ultrasound waves of acts 905 and 910. In an embodiment, controlling at least one operation of the first or second ultrasound transducer with the controller includes selecting one or more characteristics (e.g., frequency, amplitude, directionality, pulse duration, pulse sequence, etc.) of the first and second ultrasound waves prior to emitting the first and second ultrasound waves. In an embodiment, controlling at least one operation of the first or second ultrasound transducer with the controller includes changing one or more characteristics of the first or second ultrasound wave. In an embodiment, when detecting one or more characteristics includes detecting blood pressure, controlling at least one operation of the first or second ultrasound transducer with the controller includes controllably and selectively reducing or increasing the blood pressure of the subject with the acoustic wave.

The method 900 can include emitting a third ultrasound wave in a third direction from a third ultrasound transducer. The third ultrasound wave can exhibit a third frequency that is different than at least one the first frequency, the second frequency, or the frequency of the acoustic wave. In an embodiment, emitting the third ultrasound wave in a third direction can include non-linearly interacting the third ultrasound wave with at least one of the first ultrasound wave, the second ultrasound wave, or the acoustic wave. In an embodiment, emitting the third ultrasound wave in a third direction can include at least one of emitting an unfocused third ultrasound wave, not intersecting the third ultrasound wave with the first ultrasound wave, the second ultrasound wave, and the acoustic wave, or constructively or destructively interacting the third ultrasound wave with at least one of the first ultrasound wave, the second ultrasound wave, or the acoustic wave.

The reader will recognize that the state of the art has progressed to the point where there is little distinction left between hardware and software implementations of aspects of systems; the use of hardware or software is generally (but not always, in that in certain contexts the choice between hardware and software can become significant) a design choice representing cost vs. efficiency tradeoffs. The reader will appreciate that there are various vehicles by which processes or systems or other technologies described herein can be effected (e.g., hardware, software, or firmware), and that the preferred vehicle will vary with the context in which the processes or systems or other technologies are deployed. For example, if an implementer determines that speed and accuracy are paramount, the implementer can opt for a mainly hardware or firmware vehicle; alternatively, if flexibility is paramount, the implementer can opt for a mainly software implementation; or, yet again alternatively, the implementer can opt for some combination of hardware, software, or firmware. Hence, there are several possible vehicles by which the processes or devices or other technologies described herein can be effected, none of which is inherently superior to the other in that any vehicle to be utilized is a choice dependent upon the context in which the vehicle will be deployed and the specific concerns (e.g., speed, flexibility, or predictability) of the implementer, any of which can vary. The reader will recognize that optical aspects of implementations will typically employ optically-oriented hardware, software, and or firmware.

The foregoing detailed description has set forth various embodiments of the devices or processes via the use of block diagrams, flowcharts, or examples. Insofar as such block diagrams, flowcharts, or examples contain one or more functions or operations, it will be understood by those within the art that each function or operation within such block diagrams, flowcharts, or examples can be implemented, individually or collectively, by a wide range of hardware, software, firmware, or virtually any combination thereof. In an embodiment, several portions of the subject matter described herein can be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry or writing the code for the software and or firmware would be well within the skill of one of skill in the art in light of this disclosure. In addition, the reader will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a Compact Disc (CD), a Digital Video Disk (DVD), a digital tape, a computer memory, etc.; and a transmission type medium such as a digital or an analog communication medium (e.g., a fiber optic cable, a waveguide, a wired communications link, a wireless communication link, etc.).

In a general sense, the various embodiments described herein can be implemented, individually or collectively, by various types of electro-mechanical systems having a wide range of electrical components such as hardware, software, firmware, or virtually any combination thereof; and a wide range of components that can impart mechanical force or motion such as rigid bodies, spring or torsional bodies, hydraulics, and electro-magnetically actuated devices, or virtually any combination thereof. Consequently, as used herein “electro-mechanical system” includes, but is not limited to, electrical circuitry operably coupled with a transducer (e.g., an actuator, a motor, a piezoelectric crystal, etc.), electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes or devices described herein, or a microprocessor configured by a computer program which at least partially carries out processes or devices described herein), electrical circuitry forming a memory device (e.g., forms of random access memory), electrical circuitry forming a communications device (e.g., a modem, communications switch, or optical-electrical equipment), and any non-electrical analog thereto, such as optical or other analogs. Those skilled in the art will also appreciate that examples of electro-mechanical systems include but are not limited to a variety of consumer electrical systems, as well as other systems such as motorized transport systems, factory automation systems, security systems, and communication/computing systems. Those skilled in the art will recognize that electro-mechanical as used herein is not necessarily limited to a system that has both electrical and mechanical actuation except as context can dictate otherwise.

In a general sense, the various aspects described herein which can be implemented, individually or collectively, by a wide range of hardware, software, firmware, or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a computing device configured by a computer program (e.g., a general purpose computer configured by a computer program which at least partially carries out processes or devices described herein, or a microprocessor configured by a computer program which at least partially carries out processes or devices described herein), electrical circuitry forming a memory device (e.g., forms of random access memory), or electrical circuitry forming a communications device (e.g., a modem, communications switch, or optical-electrical equipment). The subject matter described herein can be implemented in an analog or digital fashion or some combination thereof.

This disclosure has been made with reference to various example embodiments. However, those skilled in the art will recognize that changes and modifications can be made to the embodiments without departing from the scope of the present disclosure. For example, various operational steps, as well as components for carrying out operational steps, can be implemented in alternate ways depending upon the particular application or in consideration of any number of cost functions associated with the operation of the system; e.g., one or more of the steps can be deleted, modified, or combined with other steps.

Additionally, as will be appreciated by one of ordinary skill in the art, principles of the present disclosure, including components, can be reflected in a computer program product on a computer-readable storage medium having computer-readable program code means embodied in the storage medium. Any tangible, non-transitory computer-readable storage medium can be utilized, including magnetic storage devices (hard disks, floppy disks, and the like), optical storage devices (CD-ROMs, DVDs, Blu-ray discs, and the like), flash memory, or the like. These computer program instructions can be loaded onto a general purpose computer, special purpose computer, or other programmable data processing apparatus to produce a machine, such that the instructions that execute on the computer or other programmable data processing apparatus create a means for implementing the functions specified. These computer program instructions can also be stored in a computer-readable memory that can direct a computer or other programmable data processing apparatus to function in a particular manner, such that the instructions stored in the computer-readable memory produce an article of manufacture, including implementing means that implement the function specified. The computer program instructions can also be loaded onto a computer or other programmable data processing apparatus to cause a series of operational steps to be performed on the computer or other programmable apparatus to produce a computer-implemented process, such that the instructions that execute on the computer or other programmable apparatus provide steps for implementing the functions specified.

The herein described components (e.g., steps), devices, and objects and the discussion accompanying them are used as examples for the sake of conceptual clarity. Consequently, as used herein, the specific exemplars set forth and the accompanying discussion are intended to be representative of their more general classes. In general, use of any specific exemplar herein is also intended to be representative of its class, and the non-inclusion of such specific components (e.g., steps), devices, and objects herein should not be taken as indicating that limitation is desired.

With respect to the use of substantially any plural or singular terms herein, the reader can translate from the plural to the singular or from the singular to the plural as is appropriate to the context or application. The various singular/plural permutations are not expressly set forth herein for sake of clarity.

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically mateable or physically interacting components or wirelessly interactable or wirelessly interacting components or logically interacting or logically interactable components.

In some instances, one or more components can be referred to herein as “configured to.” The reader will recognize that “configured to” can generally encompass active-state components or inactive-state components or standby-state components, unless context requires otherwise.

While particular aspects of the present subject matter described herein have been shown and described, it will be apparent to those skilled in the art that, based upon the teachings herein, changes and modifications can be made without departing from the subject matter described herein and its broader aspects and, therefore, the appended claims are to encompass within their scope all such changes and modifications as are within the true spirit and scope of the subject matter described herein. Furthermore, it is to be understood that the invention is defined by the appended claims. In general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims can contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” or “an” should typically be interpreted to mean “at least one” or “one or more”); the same holds true for the use of definite articles used to introduce claim recitations. In addition, even if a specific number of an introduced claim recitation is explicitly recited, such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense the convention (e.g., “a system having at least one of A, B, and C” would include but not be limited to systems that have A alone, B alone, C alone, A and B together, A and C together, B and C together, or A, B, and C together, etc.). In those instances where a convention analogous to “at least one of A, B, or C, etc.” is used, in general such a construction is intended in the sense the convention (e.g., “a system having at least one of A, B, or C” would include but not be limited to systems that have A alone, B alone, C alone, A and B together, A and C together, B and C together, or A, B, and C together, etc.). Virtually any disjunctive word or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

With respect to the appended claims, the recited operations therein can generally be performed in any order. Examples of such alternate orderings can include overlapping, interleaved, interrupted, reordered, incremental, preparatory, supplemental, simultaneous, reverse, or other variant orderings, unless context dictates otherwise. With respect to context, even terms like “responsive to,” “related to,” or other past-tense adjectives are generally not intended to exclude such variants, unless context dictates otherwise.

While various aspects and embodiments have been disclosed herein, the various aspects and embodiments disclosed herein are for purposes of illustration and are not intended to be limiting, with the true scope and spirit being indicated by the following claims.

1. A nerve modulation device, comprising:
a first ultrasound transducer configured to emit a first ultrasound wave exhibiting a first frequency, the first ultrasound transducer positionable on or in a subject, the first ultrasound transducer configured to emit the first ultrasound wave in a first direction when the first ultrasound transducer is positioned on or in the subject;
a second ultrasound transducer configured to emit a second ultrasound wave exhibiting a second frequency that is different than the first frequency, the second ultrasound transducer positionable on or in the subject, the second ultrasound transducer configured to emit the second ultrasound wave in a second direction when the second ultrasound transducer is positioned on or in the subject, wherein the second direction is selected to intersect the second ultrasound wave with the first ultrasound wave at an intersection site at or near a selected nerve;
a controller operably coupled to the first ultrasound transducer and the second ultrasound transducer, the controller configured to direct the first ultrasound transducer and the second ultrasound transducer to selectively and controllably emit the first ultrasound wave and the second ultrasound wave.

2. (canceled)

3. (canceled)

4. The nerve modulation device of claim 1, wherein the first frequency of the first ultrasound wave and the second frequency of the second ultrasound wave are selected to non-linearly interact to form an acoustic wave exhibiting a frequency that is less than the first frequency and the second frequency.

5. (canceled)

6. (canceled)

7. (canceled)

8. (canceled)

9. (canceled)

10. (canceled)

11. The nerve modulation device of claim 4, wherein the first ultrasound transducer and the second ultrasound transducer are configured to pulse the first ultrasound wave and the second ultrasound wave.

12. (canceled)

13. (canceled)

14. (canceled)

15. (canceled)

16. (canceled)

17. (canceled)

18. The nerve modulation device of claim 1, further comprising at least one attachment device configured to maintain the at least one of the first ultrasound transducer or the second ultrasound transducer on an external surface of the subject.

19. The nerve modulation device of claim 18, wherein the at least one attachment device includes at least one wearable apparatus.

20. The nerve modulation device of claim 18, wherein the at least one attachment device includes a suction cup or an adhesive.

21. (canceled)

22. The nerve modulation device of claim 18, further comprising at least one actuator coupled to the at least one attachment device that is configured to controllably switch the nerve modulation device between a tight state and a loose state, wherein a force provided by the at least one attachment device is greater when the nerve modulation device is in the tight state than when the nerve modulation device is in the loose state.

23. (canceled)

24. (canceled)

25. The nerve modulation device of claim 1, wherein the at least one of the first ultrasound transducer or the second ultrasound transducer includes an ultrasound array configured to focus the first ultrasound wave or the second ultrasound wave, respectively, wherein the ultrasound array comprises a plurality of ultrasound sources.

26. The nerve modulation device of claim 1, wherein the at least one of the first ultrasound transducer or the second ultrasound transducer comprises an acoustic lens configured to focus the first ultrasound wave or the second ultrasound wave, respectively.

27. (canceled)

28. The nerve modulation device of claim 1, wherein at least one of the first ultrasound transducer or the second ultrasound transducer includes an actuator that is configured to controllably change the first direction or the second direction, respectively, responsive to direction from the controller.

29. The nerve modulation device of claim 1, wherein the controller is configured to select the first frequency of the first ultrasound wave or the second frequency of the second ultrasound wave.

30. The nerve modulation device of claim 1, further comprising at least one sensor configured to detect one or more characteristics of the subject, the at least one sensor communicably coupled to the controller and configured to transmit one or more sensing signals to the controller responsive to detecting the one or more characteristics of the subject.

31. (canceled)

32. The nerve modulation device of claim 30, wherein the at least one sensor includes at least one nerve signal sensor.

33. The nerve modulation device of claim 30, wherein the at least one sensor includes at least one physiological sensor.

34. (canceled)

35. The nerve modulation device of claim 33, wherein the at least one physiological sensor includes at least one blood pressure sensor, image sensor, optical sensor, electromyograph, or accelerometer.

36. The nerve modulation device of claim 1, further comprising at least one sensor configured to detect one or more characteristics of an acoustic wave formed by non-linearly interacting the first ultrasound wave and the second ultrasound wave, the at least one sensor communicably coupled to the controller and configured to transmit one or more sensing signals to the controller responsive to detecting the one or more characteristics of the acoustic wave.

37. The nerve modulation device of claim 1, further comprising a third ultrasound transducer configured to emit a third ultrasound wave exhibiting a third frequency that is different than at least one of the first frequency or the second frequency, the third ultrasound transducer positionable on or in the subject, the third ultrasound transducer is configured to emit the third ultrasound wave in a third direction when the third ultrasound transducer is positioned on or in the subject.

38. (canceled)

39. (canceled)

40. A method to modulate a selected nerve of a subject, the method comprising:
emitting a first ultrasound wave in a first direction from a first ultrasound transducer, the first ultrasound wave exhibiting a first frequency;
emitting a second ultrasound wave in a second direction from a second ultrasound transducer, the second ultrasound wave exhibiting a second frequency that is different than the first frequency;
intersecting the first ultrasound wave and the second ultrasound wave at an intersection site that is within the subject and at or near a selected nerve;
responsive to intersecting the first ultrasound wave and the second ultrasound wave, non-linearly interacting the first ultrasound wave with the second ultrasound wave to form an acoustic wave having a frequency that is less than the first frequency and the second frequency; and
exposing the selected nerve of the subject to the acoustic wave.

41. The method of claim 40, wherein emitting a first ultrasound wave and emitting a second ultrasound wave includes pulsing the first ultrasound wave and the second ultrasound wave.

42. (canceled)

43. (canceled)

44. (canceled)

45. (canceled)

46. The method of claim 40, further comprising, prior to emitting the first ultrasound wave, positioning the first ultrasound transducer against an external surface of the subject and maintaining the first ultrasound transducer against the external surface using at least one attachment device.

47. (canceled)

48. (canceled)

49. (canceled)

50. The method of claim 46, further comprising controllably switching the nerve modulation device between a tight state and a loose state with at least one actuator coupled to the at least one attachment device, wherein a force provided by the at least one attachment device is greater when the at least one attachment device is in the tight state than when the at least one attachment device is in the loose state.

51. The method of claim 46, wherein positioning the first ultrasound transducer against an external surface of the subject includes positioning an ultrasound-conductive material between the first ultrasound transducer and the external surface of the subject.

52. (canceled)

53. (canceled)

54. The method of claim 46, wherein positioning the first ultrasound transducer against an external surface of the subject includes positioning the first ultrasound transducer on a neck of the subject.

55. (canceled)

56. The method of claim 40, wherein emitting a first ultrasound wave includes focusing the first ultrasound wave towards the intersection site.

57. The method of claim 56, wherein emitting a second ultrasound wave includes focusing the second ultrasound wave towards the intersection site.

58. (canceled)

59. (canceled)

60. The method of claim 40, wherein emitting a first ultrasound wave includes emitting the first ultrasound wave in an unfocused and uniform manner towards the intersection site, wherein the first ultrasound transducer is positioned from the intersection site by a distance that is less than a Fresnel length of the first ultrasound wave.

61. The method of claim 40, wherein the intersection site is at the selected nerve of the subject.

62. The method of claim 40, wherein the intersection site is near the selected nerve of the subject.

63. (canceled)

64. The method of claim 40, wherein the frequency of the acoustic wave is about 20 Hz to about 20 kHz.

65. The method of claim 40, wherein the frequency of the acoustic wave is about 20 kHz to about 1 MHz.

66. The method of claim 40, wherein the frequency of the acoustic wave is about 2 Hz to about 20 Hz.

67. The method of claim 40, wherein exposing the selected nerve of the subject to the acoustic wave includes exposing a nerve including baroreceptors to the acoustic wave.

68. The method of claim 40, wherein exposing the selected nerve of the subject to the acoustic wave includes providing an excitatory effect to the selected nerve.

69. The method of claim 40, wherein exposing the selected nerve of the subject to the acoustic wave includes providing an inhibitory effect to the selected nerve.

70. The method of claim 40, further comprising:
detecting one or more characteristics with at least one sensor;
responsive to detecting the one or more characteristics, transmitting one or more sensing signals from the at least one sensor and receiving the one or more sensing signals at a controller; and
responsive to receiving the one or more sensing signals at the controller, controlling at least one operation of at least one of the first ultrasound transducer or the second ultrasound transducer with the controller.

71. (canceled)

72. The method of claim 70, wherein detecting one or more characteristics of the subject with at least one sensor includes detecting one or more nerve signals with a nerve signal sensor.

73. The method of claim 70, wherein detecting one or more characteristics of the subject with at least one sensor includes detecting one or more physiological characteristics of the subject with a physiological sensor.

74. The method of claim 73, wherein detecting one or more physiological characteristics of the subject with a physiological sensor includes detecting tremors or lack of tremors in the subject with an image sensor, optical sensor, electromyograph, or accelerometer.

75. The method of claim 73, wherein detecting one or more physiological characteristics of the subject with a physiological sensor includes detecting blood pressure of the subject with a blood pressure sensor.

76. The method of claim 75, wherein responsive to detecting blood pressure of the subject with a blood pressure sensor, controlling at least one operation of at least one of the first ultrasound transducer or the second ultrasound transducer with the controller includes controllably and selectively reducing the blood pressure of the subject using the acoustic wave.

77. (canceled)

78. (canceled)

79. (canceled)

80. (canceled)

81. The method of claim 70, wherein controlling at least one operation of at least one of the first ultrasound transducer or the second ultrasound transducer with the controller includes changing a directionality of at least one of the first ultrasound wave or the second ultrasound wave.

82. The method of claim 70, wherein controlling at least one operation of at least one of the first ultrasound transducer or the second ultrasound transducer with the controller includes changing at least one of the first frequency of the first ultrasound wave or the second frequency of the second ultrasound wave.

83. (canceled)

84. The method of claim 40, further comprising emitting a third ultrasound wave in a third direction from a third ultrasound transducer, the third ultrasound wave exhibiting a third frequency that is different than at least one the first frequency, the second frequency, or the frequency of the acoustic wave.

85. The method of claim 84, wherein emitting a third ultrasound wave in a third direction includes non-linearly interacting the third ultrasound wave with at least one of the first ultrasound wave, the second ultrasound wave, or the acoustic wave.

86. (canceled)

87. A method to modulate a selected nerve of a subject, the method comprising:
positioning a first ultrasound transducer and a second ultrasound transducer against an external surface of the subject;
detecting one or more characteristics of the subject with at least one sensor;
responsive to detecting the one or more characteristics of the subject, transmitting one or more sensing signals from the at least one sensor and receiving the one or more sensing signals at a controller;
responsive to receiving the one or more sensing signals at the controller, under the direction of the controller:
emitting a focused first ultrasound wave in a first direction from the first ultrasound transducer, the first focused ultrasound wave exhibiting a first frequency;
emitting a second focused ultrasound wave in a second direction from the second ultrasound transducer, the second focused ultrasound wave exhibiting a second frequency that is different than the first frequency; and
intersecting the first focused ultrasound wave and the second focused ultrasound wave at an intersection site that is in the subject;
responsive to intersecting the first focused ultrasound wave and the second focused ultrasound wave, non-linearly interacting the first focused ultrasound wave with the second focused ultrasound wave to form an acoustic wave having a frequency that is less than the first frequency and the second frequency; and
exposing the selected nerve of the subject to the acoustic wave.
